(19) United States
(12) Patent Application Publication
(10) Pub. No.: US 2026/0275599 A1
(43) Pub. Date: Sep. 17, 2026

(54) FLASH EVAPORATION MATERIAL WITH LOW HAZE ATTENUATION RATE

(71) Applicant: Kingwills Advanced Materials Co. Ltd., Nantong, Jiangsu Province (CN)

(72) Inventor: Kongmeng Ye, Nantong, Jiangsu Province (CN)

(21) Appl. No.: 18/872,416

(22) PCT Filed: Oct. 7, 2023
(86) PCT No.: PCT/CN2023/123169

§ 371 Date: Dec. 6, 2024

(30) Foreign Application Priority Data
Jul. 14, 2023 (CN) 202310863987.9

(51) Int. Cl. D04H 3/007; D01D 1/02; D01D 5/11

(52) U.S. Cl. CPC D04H 3/007; D01D 1/02; D01D 5/11; D10B 2321/021

(57) ABSTRACT

The present disclosure relates to a flash evaporation material with a low haze attenuation rate, wherein the raw material of the flash evaporation material comprises polyethylene, and a grammage of a flash spun non-woven fabric is greater than 35 g/m², and a compression specific power P of the flash evaporation material is greater than 0.07 gf·cm/cm²; the compression specific power is tested by a KES style tester, and the compression specific power is tested by FB3; a haze attenuation rate ΔW of the flash evaporation material is 0.3%-1.5%; ΔW=(W₀−W₅)/W₀*100%; a rate attenuation rate ΔS of a heat shielding of the flash evaporation material is 7%-15%; ΔS=(S₀−S₅)/S₀*100%. The present application utilizes the modification of a spinning raw material to enhance the performance of the flash evaporation material and expand its application.

### TECHNICAL FIELD

The present disclosure belongs to the field of flash evaporation technology, and in particular relates to a flash evaporation material with a low haze attenuation rate.

### BACKGROUND

Flash evaporation spinning involves dissolving PE, PP and other similar polymers in a certain spinning solvent to produce a spinning solution, which is then ejected from the spinneret. Due to the rapid evaporation of the solvent, the polymer is re-solidified into fibers, which are adsorbed onto a lapping curtain to directly form a web, and then are prepared into a flash evaporation material through hot pressing and winding processes. The current polymer raw material for flash evaporation method is PE material, and a polyethylene non-woven fabric produced by flash evaporation method has many excellent properties, such as excellent waterproof and breathable properties; the sheet has excellent strength, tear resistance, puncture resistance, and rupture resistance; the sheet generally does not fuzz or produce dust; and it has excellent performance over a wide temperature range, etc. Therefore, the polyethylene non-woven fabric produced by flash evaporation method is widely used in packaging materials, protective clothing, cover cloths, and printing substrates.

Chinese Patent Publication No. CN114687057A relates to a flash evaporation sheet and an application thereof, wherein raw materials of the flash evaporation sheet are polyethylene and a modifier; an attenuation value ΔC of a bending length of the flash evaporation sheet is 0.1-0.4; ΔC=1−C2/C1; C1 is the bending length of the flash evaporation sheet that has not undergone aging treatment; C2 is the bending length of the flash evaporation sheet after aging treatment; and the bending length C2 of the flash evaporation sheet after aging treatment is 4 cm-10 cm. While maintaining brightness, the flash evaporation sheet of the present application also has a lower decrease in bending length, which can extend the service life of the product and expand the scope of application.

Chinese Patent Publication No. CN114687069A relates to a multifunctional polymer non-woven fabric and a fabric thereof, wherein an attenuation rate ▴E of an elongation at break is 0.20-0.50; ▴E=1−E2/E1; E1 is the elongation at break of the multifunctional polymer non-woven fabric that has not undergone aging test treatment; E2 is the elongation at break of the multifunctional polymer non-woven fabric after aging test treatment; and the value of E2 is less than 0.5. The process of the present application is simple and widely used.

Chinese Patent Publication No. CN114687068A relates to an improved flash evaporation non-woven fabric. The present disclosure aims at the problem in the prior art that a brightness of the flash evaporation non-woven fabric will decrease after a period of use, and provides an improved flash evaporation non-woven fabric. A loss rate ▴F of a D65 fluorescence brightness of the improved flash evaporation non-woven fabric is 0.20-0.30; wherein ▴F=1−F2/F1; F1 is the D65 fluorescence brightness of the non-woven fabric that has not undergone aging treatment; F2 is the D65 fluorescence brightness of the non-woven fabric after aging treatment; and the process conditions for aging treatment are: an irradiance within the wavelength range of 300 nm-400 nm is 60 w/m2±2 w/m², a black mark temperature is 65° C.±2° C., an air temperature in a test chamber is 38° C.±3° C., a relative humidity is 50%±10%, and a drying time is 1440 hours. The flash evaporation non-woven fabric of the present disclosure can maintain a high brightness after a period of use.

Chinese Patent Publication No. CN114108112A relates to a polyethylene sheet, wherein an aging rate ΔS of a tensile strength is 0.10-0.30; ΔS=1−S2/S1; S1 is the tensile strength of the non-woven fabric that has not undergone aging treatment, unit kN/m; S2 is the tensile strength of the non-woven fabric after aging treatment, unit kN/m; and the process conditions for aging treatment are: an irradiance within a wavelength range of 300 nm-400 nm is 60 w/m2±2 w/m², a black mark temperature is 65° C.±2° C., an air temperature in a test chamber is 38° C.±3° C., a relative humidity is 50%±10%, and a drying time is 1440 hours. The product of the present disclosure still has a good tensile strength after aging, thereby extending a service life of the product.

Chinese Patent Publication No. CN115537959A relates to a composite material, wherein a raw material of the composite material contains polyethylene; a shrinkage rate R is 0.2-0.7; a standard heat shrinkage strength or is 0.5 N/mm²-3.5 N/mm²; and an anti-mold level is less than level 2. The present application has a good shrinkage performance and better antibacterial and anti-mold effects, so it has a wide range of applications.

Chinese Patent Publication No. CN109154138A relates to a composite material comprising a non-woven vapor permeable sheet, a composite laminate, which comprises at least one water vapor permeable non-woven sheet having first and second surfaces and a fluorinated polymer coating on the first surface of the sheet, wherein (i) the fluorinated polymer is coated in an amount such that a total fluorine content of the coated non-woven sheet is present in an amount from 0.05 gsm to no greater than 0.4 gsm, and (ii) the composite laminate exhibits at least 60% retained hydrohead after exposure to wet wood.

U.S. Patent Publication No. US20160138197A1 relates to a flash spun plexifilamentary protofilament and a flash spun plexifilamentary fiber bundle with a BET surface area of less than 12 m²/g and a crushing value of at least 0.9 mm/g, wherein the fiber bundle mainly comprises fibers formed from polyethylene, the fibers have a summary crystallinity index of less than 55%, and the fiber bundle has a sheet made of an elongation at break greater than 55%.

U.S. Pat. No. 8,048,513 flash spun sheet relates to an improved plexifilamentary sheet for use in a protective clothing and a filter media, wherein the material is composed of substantially continuous polyethylene plexifilamentary fiber bundles and has a Frazier permeability, normalized to 1.0 oz/yd², of at least 2 cfm/ft².

In the prior art, there are few studies on a heat shielding performance and a haze performance of the flash evaporation material, and there is a problem that the heat shielding performance and the haze performance of the flash evaporation material decrease after a period of use.

### SUMMARY OF THE INVENTION

The purpose of the present disclosure is to provide a flash evaporation material with a low haze attenuation rate to solve the above-mentioned problems.

In order to achieve the above-mentioned objects, the present disclosure adopts the following technical solutions:
a flash evaporation material with a low haze attenuation rate, wherein a raw material of the flash evaporation material comprises polyethylene, and a grammage of a flash spun non-woven fabric is greater than 35 g/m²,
a compression specific power P of the flash evaporation material is greater than 0.07 gf·cm/cm²;
the compression specific power is tested by a KES style tester, and the compression specific power is tested by FB3;
a haze attenuation rate ΔW of the flash evaporation material is 0.3%-1.5%;

$$△ W = ( {W}_{0} - {W}_{5} ) / {W}_{0} * 100 \% ;$$

an attenuation rate ΔS of a heat shielding rate of the flash evaporation material is 7%-15%;

$$△ S = ( {S}_{0} - {S}_{5} ) / {S}_{0} * 100 \%$$

tests for a heat shielding rate, a haze and a transverse tensile strength:
(1) placing a sample at 25° C. and a relative humidity of 65% for 24 hours; then measuring and recording a shading index thereof as an initial heat shielding rate S₀, measuring and recording a haze thereof as an initial haze W₀, and measuring and recording a transverse tensile strength as a transverse tensile strength CTS₀;
(2) then exposing the sample to a dry and hot atmosphere of 90° C. for 6 hours, and then cooling at 25° C. and a relative humidity of 65% for 24 hours;
(3) repeating the operation of step (2), and after treatment for 4 times, measuring and recording a shading index thereof as a final heat shielding rate S₅, measuring and recording a haze thereof as a final haze W₅, and measuring and recording a transverse tensile strength thereof as a final transverse tensile strength CTS₅;
the haze is tested according to the national standard GB/T2410-2008; and
the heat shielding rate is measured according to the national standard GB/T41560-2022.

Tensile strength is determined according to the national standard GB/T 12914-2018. Tensile strength is a maximum tension that a unit width specimen can withstand before fracture under specified test conditions.

The grammage of the flash spun non-woven fabric is less than 75 g/m².

The grammage of the flash spun non-woven fabric is less than 65 g/m².

The compression specific work P of the flash spun non-woven fabric is greater than 0.15 gf·cm/cm².

The compression specific work P of the flash spun non-woven fabric is less than 2 gf·cm/cm².

The compression specific work P of the flash spun non-woven fabric is less than 1 gf·cm/cm².

The haze attenuation rate ΔW of the flash evaporation material is 0.6%-0.9%.

The haze attenuation rate ΔW of the flash evaporation material is 0.9%-1.3%.

The attenuation rate of the heat shielding rate of the flash evaporation material is 7%-10%.

The attenuation rate of the heat shielding rate of the flash evaporation material is 10%-14%.

The attenuation rate ΔCTS of the transverse tensile strength of the flash evaporation material is 2%-10%;

$$△ CTS = ( {CTS}_{0} - {CTS}_{5} ) / {CTS}_{0} * 100 \% ;$$

and
the transverse tensile strength CTS₀ is greater than 1.4 KN/m.

The attenuation rate ΔCTS of the transverse tensile strength of the flash evaporation material is 3%-5%.

The attenuation rate ΔCTS of the transverse tensile strength of the flash evaporation material is 5%-7%.

The attenuation rate ΔCTS of the transverse tensile strength of the flash evaporation material is 7%-9%.

A preparation method of a flash evaporation material with a low haze attenuation rate, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 3%-6%;
a preparation method of the porous carbon material loaded with zinc oxide, which includes specific technical steps:
(1) preparation of a solution of silica nanoparticles surface-coated with polyvinyl alcohol:
adding silica nanoparticles to a deionized water for ultrasonic dispersion to obtain a dispersion, then adding a polyvinyl alcohol powder to the dispersion while heating and stirring, controlling a heating temperature to 80° C.-100° C. for dissolution and dispersion, waiting for the polyethylene to be dissolved and dispersed, and performing a reaction for 36-60 hours after the polyvinyl alcohol solution is dissolved, and then removing the undissolved polyvinyl alcohol by filtration to obtain a coating solution, which is a solution of silica nanoparticles surface-coated with polyvinyl alcohol;
a mass ratio of the silica nanoparticles to the polyvinyl alcohol powder is 1:9-1:12;
(2) preparation of a zinc-containing porous carbon precursor dispersion
further adding the solution of silica nanoparticles surface-coated with polyvinyl alcohol prepared in step (1) to a zinc nitrate aqueous solution to continue a first-order reaction, thus making zinc and polyvinyl alcohol perform a chelation reaction and being loaded on the surface of the silica nanoparticles, then using an alkali to slowly adjust a pH value of a mixed solution to 11-12, and continuing a second-order reaction under alkali conditions to obtain the zinc-containing porous carbon precursor dispersion;
a first-order reaction time is 30-48 hours, and a reaction temperature is 60° C.-80° C.;
the alkali can specifically be 0.1 mol/L sodium hydroxide solution or potassium hydroxide solution;
a second-order reaction is performed under alkaline conditions, and a reaction time is 4-8 hours;
in this step, an internal silicon dioxide is etched, and at the same time, internal zinc ions generate a zinc hydroxide precipitation in an alkaline solution, achieving simultaneous silicon dioxide etching and zinc hydroxide precipitation;
(3) preparation of a porous carbon material loaded with zinc oxide:
finally, separating the zinc-containing porous carbon precursor dispersion in step (2) by centrifugation, and taking a sediment from a lower layer to obtain a zinc-containing porous carbon precursor; performing aerobic calcination on the zinc-containing porous carbon precursor in the lower layer aerobically at 180° C.-200° C. for 15-30 minutes, and then rapidly heating up to 440° C.-450° C. within 1-5 minutes, performing anaerobic calcination for 45-60 minutes, and obtaining the porous carbon material loaded with zinc oxide after natural cooling.

During the calcination process, performing aerobic calcination first, due to the aerobic pre-oxidation carbonization of polyvinyl alcohol structure at 180° C., and the oxidation decomposition of zinc hydroxide to produce zinc oxide, and then transferring to high-temperature anaerobic carbonization, and making a carbon material formed in the anaerobic environment perform oxidation-reduction reaction with zinc oxide, ultimately producing an excellent zinc oxide, avoiding the dispersion problem of conventional blending and adding, and making zinc oxide generate on the surface of the carbon material to replace a reduced carbon material, forming a relatively stable porous carbon structure doped with zinc oxide. The porous carbon structure doped with zinc oxide is filled in gaps between flash evaporation fibers of the flash evaporation material, reducing the transmission of light and increasing the emission and refraction of light. Zinc oxide has a good emission performance, thereby improving a heat shielding performance of the product.

The spinning solvent includes a surfactant and a solvent; and
the surfactant in the spinning solvent is 500 ppm-2000 ppm.

The surfactant is polyvinylpyrrolidone.

The solvents include aromatic hydrocarbons, aliphatic hydrocarbons, cycloaliphatic hydrocarbons, unsaturated hydrocarbons, halogenated hydrocarbons, and their analogues.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.
a temperature of the flash evaporation spinning is 200° C.-220° C.; and
a temperature of the hot pressing is 105° C.-115° C.

Compared with the prior art, the present disclosure has the following positive effects:
the present disclosure has found through experiments that the haze attenuation rate and the heat shielding rate attenuation rate of the flash evaporation material are at least closely related to factors such as the content of the porous carbon material loaded with zinc oxide in the spinning raw material and the content of the surfactant in the spinning solvent, and provides a novel preparation process for the flash evaporation material, which enables the flash evaporation material to have a lower haze attenuation rate and a lower attenuation rate of the heat shielding rate, thus overcoming the problem of significant decline in the heat shielding performance and the haze performance of the flash evaporation material after a period of use in the prior art.

### DETAILED DESCRIPTION OF THE INVENTION

The present disclosure will be described in further detail below in conjunction with specific embodiments.

### Example 1

This embodiment provides a flash evaporation material with a low haze attenuation rate, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 3%;
a preparation method of the porous carbon material loaded with zinc oxide, which includes specific technical steps:
(1) preparation of a solution of silica nanoparticles surface-coated with polyvinyl alcohol:
adding silica nanoparticles to a deionized water for ultrasonic dispersion to obtain a dispersion, then adding a polyvinyl alcohol powder to the dispersion while heating and stirring, controlling a heating temperature to 80° C.-100° C. for dissolution and dispersion, waiting for the polyethylene to be dissolved and dispersed, and performing a reaction for 36-60 hours after the polyvinyl alcohol solution is dissolved, and then removing the undissolved polyvinyl alcohol by filtration to obtain a coating solution, which is a solution of silica nanoparticles surface-coated with polyvinyl alcohol;
a mass ratio of the silica nanoparticles to the polyvinyl alcohol powder is 1:9;
(2) preparation of a zinc-containing porous carbon precursor dispersion
further adding the solution of silica nanoparticles surface-coated with polyvinyl alcohol prepared in step (1) to a zinc nitrate aqueous solution to continue a first-order reaction, thus making zinc and polyvinyl alcohol perform a chelation reaction and being loaded on the surface of the silica nanoparticles, then using an alkali to slowly adjust a pH value of a mixed solution to 11-12, and continuing a second-order reaction under alkali conditions to obtain the zinc-containing porous carbon precursor dispersion;
a first-order reaction time is 30 hours, and a reaction temperature is 60° C.-80° C.;
the alkali can specifically be 0.1 mol/L sodium hydroxide solution or potassium hydroxide solution;
a second-order reaction is performed under alkaline conditions, and a reaction time is 5 hours;
in this step, an internal silicon dioxide is etched, and at the same time, internal zinc ions generate a zinc hydroxide precipitation in an alkaline solution, achieving simultaneous silicon dioxide etching and zinc hydroxide precipitation;
(3) preparation of a porous carbon material loaded with zinc oxide:
finally, separating the zinc-containing porous carbon precursor dispersion in step (2) by centrifugation, and taking a sediment from a lower layer to obtain a zinc-containing porous carbon precursor; performing aerobic calcination on the zinc-containing porous carbon precursor in the lower layer aerobically at 180° C.-200° C. for 15-30 minutes, and then rapidly heating up to 440° C.-450° C. within 1-5 minutes, performing anaerobic calcination for 45-60 minutes, and obtaining the porous carbon material loaded with zinc oxide after natural cooling.

The spinning solvent includes a surfactant and a solvent; and
the surfactant in the spinning solvent is 700 ppm.

The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 200° C.; and
a temperature of the hot pressing is 105° C.

A performance test data of the flash evaporation material prepared in this embodiment is shown in Table 1.

### Example 2

This embodiment provides a flash evaporation material with a low haze attenuation rate, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 4.5%;
a preparation method of the porous carbon material loaded with zinc oxide, which includes specific technical steps:
(1) preparation of a solution of silica nanoparticles surface-coated with polyvinyl alcohol:
adding silica nanoparticles to a deionized water for ultrasonic dispersion to obtain a dispersion, then adding a polyvinyl alcohol powder to the dispersion while heating and stirring, controlling a heating temperature to 80° C.-100° C. for dissolution and dispersion, waiting for the polyethylene to be dissolved and dispersed, and performing a reaction for 36-60 hours after the polyvinyl alcohol solution is dissolved, and then removing the undissolved polyvinyl alcohol by filtration to obtain a coating solution, which is a solution of silica nanoparticles surface-coated with polyvinyl alcohol;
a mass ratio of the silica nanoparticles to polyvinyl alcohol powder is 1:10.5;
(2) preparation of a zinc-containing porous carbon precursor dispersion
further adding the solution of silica nanoparticles surface-coated with polyvinyl alcohol prepared in step (1) to a zinc nitrate aqueous solution to continue a first-order reaction, thus making zinc and polyvinyl alcohol perform a chelation reaction and being loaded on the surface of the silica nanoparticles, then using an alkali to slowly adjust a pH value of a mixed solution to 11-12, and continuing a second-order reaction under alkali conditions to obtain the zinc-containing porous carbon precursor dispersion;
a first-order reaction time is 40 hours, and a reaction temperature is 60° C.-80° C.;
the alkali can specifically be 0.1 mol/L sodium hydroxide solution or potassium hydroxide solution;
a second-order reaction is performed under alkaline conditions, and a reaction time is 6 hours;
in this step, an internal silicon dioxide is etched, and at the same time, internal zinc ions generate a zinc hydroxide precipitation in an alkaline solution, achieving simultaneous silicon dioxide etching and zinc hydroxide precipitation;
(3) preparation of a porous carbon material loaded with zinc oxide:
finally, separating the zinc-containing porous carbon precursor dispersion in step (2) by centrifugation, and taking a sediment from a lower layer to obtain a zinc-containing porous carbon precursor; performing aerobic calcination on the zinc-containing porous carbon precursor in the lower layer aerobically at 180° C.-200° C. for 15-30 minutes, and then rapidly heating up to 440° C.-450° C. within 1-5 minutes, performing anaerobic calcination for 45-60 minutes, and obtaining the porous carbon material loaded with zinc oxide after natural cooling.

The spinning solvent includes a surfactant and a solvent; and
the surfactant in the spinning solvent is 1200 ppm.

The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 210° C.; and
a temperature of the hot pressing is 110° C.

A performance test data of the flash evaporation material prepared in this embodiment is shown in Table 1.

### Example 3

This embodiment provides a flash evaporation material with a low haze attenuation rate, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 6%;
a preparation method of the porous carbon material loaded with zinc oxide, which includes specific technical steps:
(1) preparation of a solution of silica nanoparticles surface-coated with polyvinyl alcohol:
adding silica nanoparticles to a deionized water for ultrasonic dispersion to obtain a dispersion, then adding a polyvinyl alcohol powder to the dispersion while heating and stirring, controlling a heating temperature to 80° C.-100° C. for dissolution and dispersion, waiting for the polyethylene to be dissolved and dispersed, and performing a reaction for 36-60 hours after the polyvinyl alcohol solution is dissolved, and then removing the undissolved polyvinyl alcohol by filtration to obtain a coating solution, which is a solution of silica nanoparticles surface-coated with polyvinyl alcohol;
a mass ratio of the silica nanoparticles to polyvinyl alcohol powder is 1:12;
(2) preparation of a zinc-containing porous carbon precursor dispersion
further adding the solution of silica nanoparticles surface-coated with polyvinyl alcohol prepared in step (1) to a zinc nitrate aqueous solution to continue a first-order reaction, thus making zinc and polyvinyl alcohol perform a chelation reaction and being loaded on the surface of the silica nanoparticles, then using an alkali to slowly adjust a pH value of a mixed solution to 11-12, and continuing a second-order reaction under alkali conditions to obtain the zinc-containing porous carbon precursor dispersion;
a first-order reaction time is 48 hours, and a reaction temperature is 60° C.-80° C.;
the alkali can specifically be 0.1 mol/L sodium hydroxide solution or potassium hydroxide solution;
a second-order reaction is performed under alkaline conditions, and a reaction time is 8 hours;
in this step, an internal silicon dioxide is etched, and at the same time, internal zinc ions generate a zinc hydroxide precipitation in an alkaline solution, achieving simultaneous silicon dioxide etching and zinc hydroxide precipitation;
(3) preparation of a porous carbon material loaded with zinc oxide:
finally, separating the zinc-containing porous carbon precursor dispersion in step (2) by centrifugation, and taking a sediment from a lower layer to obtain a zinc-containing porous carbon precursor; performing aerobic calcination on the zinc-containing porous carbon precursor in the lower layer aerobically at 180° C.-200° C. for 15-30 minutes, and then rapidly heating up to 440° C.-450° C. within 1-5 minutes, performing anaerobic calcination for 45-60 minutes, and obtaining the porous carbon material loaded with zinc oxide after natural cooling.

The spinning solvent includes a surfactant and a solvent; and
the surfactant in the spinning solvent is 2000 ppm.

The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 220° C.; and
a temperature of the hot pressing is 115° C.

A performance test data of the flash evaporation material prepared in this embodiment is shown in Table 1.

### Comparative Example 1

This comparative example provides a flash evaporation material, which mainly includes the preparation of a spinning solution and the preparation of a flash evaporation material;
wherein,
preparation of a spinning solution: dissolving a spinning raw material in a spinning solvent to obtain a spinning solution;
the spinning raw material is polyethylene;
the spinning solvent includes a surfactant and a solvent; and
the surfactant in the spinning solvent is 1200 ppm.

The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 210° C.; and
a temperature of the hot pressing is 110° C.

A performance test data of the flash evaporation material prepared in this comparative example is shown in Table 1.

### Comparative Example 2

This comparative example provides a flash evaporation material, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 1%;
a preparation method of the porous carbon material loaded with zinc oxide is the same as in Example 2.

The spinning solvent includes a surfactant and a solvent; and
the surfactant in the spinning solvent is 1200 ppm.

The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 210° C.; and
a temperature of the hot pressing is 110° C.

A performance test data of the flash evaporation material prepared in this comparative example is shown in Table 1.

### Comparative Example 3

This comparative example provides a flash evaporation material, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 2%;
a preparation method of the porous carbon material loaded with zinc oxide is the same as in Example 2.

The spinning solvent includes a surfactant and a solvent; and
the surfactant in the spinning solvent is 1200 ppm.

The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 210° C.; and
a temperature of the hot pressing is 110° C.

A performance test data of the flash evaporation material prepared in this comparative example is shown in Table 1.

### Comparative Example 4

This comparative example provides a flash evaporation material, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 8%;
a preparation method of the porous carbon material loaded with zinc oxide is the same as in Example 2.

The spinning solvent includes a surfactant and a solvent; and
the surfactant in the spinning solvent is 1200 ppm.

The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 210° C.; and
a temperature of the hot pressing is 110° C.

A performance test data of the flash evaporation material prepared in this comparative example is shown in Table 1.

### Comparative Example 5

This comparative example provides a flash evaporation material, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 9%;
a preparation method of the porous carbon material loaded with zinc oxide is the same as in Example 2.

The spinning solvent includes a surfactant and a solvent; and
the surfactant in the spinning solvent is 1200 ppm.

The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 210° C.; and
a temperature of the hot pressing is 110° C.

A performance test data of the flash evaporation material prepared in this comparative example is shown in Table 1.

### Comparative Example 6

This comparative example provides a flash evaporation material, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 4.5%;
a preparation method of the porous carbon material loaded with zinc oxide is the same as in Example 2.

The spinning solvent is a solvent; and
the solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 210° C.; and
a temperature of the hot pressing is 110° C.

A performance test data of the flash evaporation material prepared in this comparative example is shown in Table 1.

### Comparative Example 7

This comparative example provides a flash evaporation material, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 4.5%;
a preparation method of the porous carbon material loaded with zinc oxide is the same as in Example 2.

The spinning solvent includes a surfactant and a solvent; and
the surfactant in the spinning solvent is 200 ppm.
The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 210° C.; and
a temperature of the hot pressing is 110° C.

A performance test data of the flash evaporation material prepared in this comparative example is shown in Table 1.

### Comparative Example 8

This comparative example provides a flash evaporation material, which mainly includes the preparation of a spinning solution and the preparation of a flash evaporation material;
wherein,
preparation of a spinning solution: dissolving a spinning raw material in a spinning solvent to obtain a spinning solution;
the spinning raw material includes polyethylene and a porous carbon material loaded with zinc oxide;
a mass fraction of the porous carbon material loaded with zinc oxide in the spinning raw material is 4.5%;
a preparation method of the porous carbon material loaded with zinc oxide is the same as in Example 2.

The spinning solvent includes a surfactant and a solvent; and
the surfactant in the spinning solvent is 2000 ppm.

The surfactant is polyvinylpyrrolidone.

The solvent is chloroethane, dichloromethane, 1,1-dichloro-2,2-difluoroethane, and 1,1,2,2,3,3,4,4-octafluorobutane, with a mass ratio of 4:4:1:1.

### Preparation of a Flash Evaporation Material:

making the prepared spinning solution enter into a spinning chamber for flash evaporation spinning, and obtaining a flash evaporation fiber through a nozzle of a spinning component, then performing laying and hot pressing processes to the flash evaporation fiber to obtain a flash evaporation material.

A temperature of the flash evaporation spinning is 210° C.; and
a temperature of the hot pressing is 110° C.

A performance test data of the flash evaporation material prepared in this comparative example is shown in Table 1.

TABLE 1
Performance test data of the flash evaporation material

| | P | W | S | CTS |
|---|---|---|---|---|
| | gf · cm/cm² | % | % | % |
| Example 1 | 0.10 | 1 | 15 | 10.7 |
| Example 2 | 0.15 | 0.7 | 10.8 | 4.4 |
| Example 3 | 0.22 | 0.4 | 7.9 | 2.8 |
| Comparative | 0.49 | 5 | 30 | 35 |
| Example 1 | | | | |
| Comparative | 0.36 | 3 | 22 | 22 |
| Example 2 | | | | |
| Comparative | 0.25 | 2 | 18 | 15 |
| Example 3 | | | | |
| Comparative | 0.05 | 0.31 | 7.5 | 2.5 |
| Example 4 | | | | |
| Comparative | 0.04 | 0.23 | 7.3 | 2.2 |
| Example 5 | | | | |
| Comparative | 0.35 | 5 | 25 | 20 |
| Example 6 | | | | |
| Comparative | 0.26 | 3 | 22 | 18 |
| Example 7 | | | | |
| Comparative | 0.06 | 0.3 | 7.7 | 3.7 |
| Example 8 | | | | |

Result analysis: comparing the above data, it can be seen that increasing the amount of the porous carbon material loaded with zinc oxide in the spinning raw material will reduce the haze attenuation rate and the attenuation rate of the heat shielding rate. However, after increasing to a certain amount, the decrease rate will significantly slow down. After increasing the amount of the surfactant in the spinning solvent, the haze attenuation rate and the attenuation rate of the heat shielding rate will also decrease. However, after increasing to a certain amount, the decrease rate will also significantly slow down.

The specific embodiments described herein are merely illustrative of the spirit of the present disclosure. It is apparent to those skilled in the art that various modifications, amendments and alternatives can be made to the described embodiments without departing from the spirit of the present disclosure or scope defined by the appended claims.

1. A flash evaporation material, wherein a raw material of the flash evaporation material comprises polyethylene, and a grammage of a flash spun non-woven fabric is greater than 35 g/m²,
a compression specific power (P) of the flash evaporation material is greater than 0.07 gf·cm/cm²;
the compression specific power is tested by a Kawabata Evaluation System (KES) style tester, and the compression specific power is tested by FB3;
a haze attenuation rate (ΔW) of the flash evaporation material is 0.3%-1.5%;
$$△ W = ( W 0 - W 5 ) / W 0 * 100 \% ;$$
an attenuation rate (ΔS) of a heat shielding rate of the flash evaporation material is 7%-15%;
$$△ S = ( S 0 - S 5 ) / S 0 * 100 \% ;$$
tests for the heat shielding rate, a haze and a transverse tensile strength comprise the following steps:
(1) placing a sample at 25° C. and a relative humidity of 65% for 24 hours; then measuring and recording a shading index of the sample as an initial heat shielding rate (S0), measuring and recording the haze of the sample as an initial haze (W0), and measuring and recording the transverse tensile strength of the sample as an initial transverse tensile strength (CTS0);
(2) then exposing the sample to a dry and hot atmosphere of 90° C. for 6 hours, and then cooling at 25° C. and a relative humidity of 65% for 24 hours;
(3) repeating the step (2), and after treatment for 4 times, measuring and recording the shading index of the sample as a final heat shielding rate (S5), measuring and recording the haze of the sample as a final haze (W5), and measuring and recording the transverse tensile strength of the sample as a final transverse tensile strength (CTS5);
the haze is tested according to national standard GB/T2410-2008; and
the heat shielding rate is measured according to national standard GB/T41560-2022.

2. The flash evaporation material of claim 1, wherein the grammage of the flash spun non-woven fabric is less than 75 g/m².

3. The flash evaporation material of claim 1, wherein a compression specific power (P) of the flash spun non-woven fabric is greater than 0.15 gf·cm/cm².

4. The flash evaporation material of claim 1, wherein the haze attenuation rate (ΔW) of the flash evaporation material is 0.6%-0.9%.

5. The flash evaporation material of claim 1, wherein the haze attenuation rate (ΔW) of the flash evaporation material is 0.9%-1.3%.

6. The flash evaporation material of claim 1, wherein the attenuation rate (ΔS) of the heat shielding rate of the flash evaporation material is 7%-10%.

7. The flash evaporation material of claim 1, wherein the attenuation rate (ΔS) of the heat shielding rate of the flash evaporation material is 10%-14%.

8. The flash evaporation material of claim 1, wherein an attenuation rate (ΔCTS) of the transverse tensile strength of the flash evaporation material is 2%-10%;
$$△ CTS = ( CTS 0 - CTS 5 ) / CTS 0 * 100 \% ;$$
the initial transverse tensile strength (CTS0) is greater than 1.4 KN/m.

9. The flash evaporation material of claim 8, wherein the attenuation rate (ΔCTS) of the transverse tensile strength of the flash evaporation material is 3%-5%.

10. The flash evaporation material of claim 8, wherein the attenuation rate (ΔCTS) of the transverse tensile strength of the flash evaporation material is 5%-7%.

11. The flash evaporation material of claim 1, wherein a spinning raw material comprises polyethylene and a porous carbon material loaded with zinc oxide, and a spinning solvent comprise a surfactant and a solvent.

12. The flash evaporation material of claim 11, wherein a mass fraction of the porous carbon material loaded with zinc oxide in the spinning raw material is 3%-6%; and
the surfactant in the spinning solvent is 500 ppm-2000 ppm.

13. The flash evaporation material of claim 11, wherein the porous carbon material loaded with zinc oxide is prepared by the following steps:
(1) preparing a solution of silica nanoparticles surface-coated with polyvinyl alcohol:
(2) preparing a zinc-containing porous carbon precursor dispersion by
further adding the solution of silica nanoparticles surface-coated with polyvinyl alcohol prepared in the step (1) to a zinc nitrate aqueous solution to continue a first-order reaction, thus making zinc and polyvinyl alcohol perform a chelation reaction and being loaded on a surface of the silica nanoparticles, then using an alkali to adjust a pH value of a mixed solution to 11-12, and continuing a second-order reaction under alkali conditions to obtain the zinc-containing porous carbon precursor dispersion; and
(3) preparing the porous carbon material loaded with zinc oxide by
finally, separating the zinc-containing porous carbon precursor dispersion obtained in the step (2) by centrifugation, and taking a sediment from a lower layer to obtain a zinc-containing porous carbon precursor; performing aerobic calcination on the zinc-containing porous carbon precursor in the lower layer aerobically at 180° C.-200° C. for 15-30 minutes, and then rapidly heating up to 440° C.-450° C. within 1-5 minutes, performing anaerobic calcination for 45-60 minutes, and obtaining the porous carbon material loaded with zinc oxide after natural cooling.
